(19) United States
(12) Patent Application Publication
(10) Pub. No.: US 2021/0187208 A1
(43) Pub. Date: Jun. 24, 2021

(54) MULTIFUNCTIONAL SKIN PATCH

(71) Applicant: THE GENERAL HOSPITAL CORPORATION, Boston, MA (US)

(72) Inventors: Yakir Levin, Newton, MA (US); Mathew Avram, Boston, MA (US); Richard Rox Anderson, Boston, MA (US)

(21) Appl. No.: 17/270,393

(22) PCT Filed: Aug. 23, 2019
(86) PCT No.: PCT/US19/47895

§ 371 Date: Feb. 22, 2021

Related U.S. Application Data
(60) Provisional application No. 62/722,120, filed on Aug. 23, 2018.

(51) Int. Cl. A61M 5/42; A61F 7/02; A61N 5/06; A61B 18/20; A61M 5/44

(52) U.S. Cl. CPC A61M 5/422; A61F 7/02; A61N 5/0616; A61N 2005/067; A61M 5/44; A61M 2205/3606; A61M 2205/3646; A61B 18/203

(57) ABSTRACT

A multifunctional skin patch can be used during a treatment procedure to achieve one or more safety factors, including skin cooling, pain management, laser plume control, and/or proper application of the treatment. The multifunctional skin patch includes a transparent hydrogel layer that can be applied to a patient's skin prior to application of the treatment procedure. The treatment procedure can be conducted through the transparent hydrogel layer, and the transparent hydrogel layer (with can be pre-cooled) can cool and/or control pain within the portion of the patient's skin during and/or after the treatment procedure. In some instances, the multifunctional skin patch can also include a transparent indicator layer (covering at least a portion of the transparent hydrogel layer) with dispersed indicator particles attached thereto and/or embedded within. The indicator particles are configured to change in appearance upon application of treatment procedure through the transparent indicator layer.

### RELATED APPLICATIONS

This application claims the benefit of U.S. Provisional Application No. 62/722,120, entitled “MULTIFUNCTIONAL SKIN PATCH TO ENABLE SAFE LASER TREATMENT,” filed Aug. 23, 2018. The entirety of this application is hereby incorporated by reference for all purposes.

### TECHNICAL FIELD

The present disclosure relates generally to a multifunctional skin patch and, more particularly, to using the multifunctional skin patch during a treatment procedure to achieve at least one of skin cooling, pain management, plume control, and proper application of the treatment.

### BACKGROUND

Many treatment procedures used for dermatology applications involve micro needling, injection, and/or energy-based devices (including lasers, light sources, ultrasound devices, and the like) to administer treatment to the skin. For example, lasers can be used for treatment of blood vessels pathologies (as in congenital vascular lesions and telangiectasias), elimination of brown spots (as in nevi and lentigines), hair removal, tattoo removal, skin resurfacing, treatment of scars, etc. One or more important safety factors should be considered before administering a treatment procedure to the skin (based on the treatment being administered), including: skin cooling, pain management, laser plume control, and proper application of the treatment.

### SUMMARY

The present disclosure provides a multifunctional skin patch that can be used during a treatment procedure to achieve one or more safety factors, including skin cooling, pain management, plume control, and/or proper application of the treatment.

In one aspect, the present disclosure can include a system that achieves one or more safety factors. The system can include a treatment device comprising a microneedle device, an injection device, and/or an energy delivery device. The system can also include a transparent hydrogel configured to be pre-cooled and applied to a portion of a patient's skin prior to a treatment procedure to cool and/or control pain within the portion of the patient's skin during and/or after the treatment procedure. The treatment device can be configured to apply the treatment procedure through the transparent hydrogel to the patient's skin. In some instances, the transparent hydrogel layer can be at least partially covered by a transparent indicator layer with dispersed indicator particles attached thereto and/or embedded within. The indicator particles are configured to change in appearance upon application of the treatment procedure through the transparent indicator layer.

In a further aspect, the present disclosure can include a device (a multifunctional skin patch) that can be used during and/or after a treatment procedure. The device includes a transparent hydrogel layer that can be applied to a patient's skin prior to application of the treatment procedure. The treatment procedure can be conducted through the transparent hydrogel layer, and the transparent hydrogel layer (with can be pre-cooled) can cool and/or control pain within the portion of the patient's skin during and/or after the treatment procedure. In some instances, the multifunctional skin patch can also include a transparent indicator layer (covering at least a portion of the transparent hydrogel layer) with dispersed indicator particles attached thereto and/or embedded within. The indicator particles are configured to change in appearance upon application of the treatment procedure through the transparent indicator layer.

In yet another aspect, the present disclosure can include a method for achieving one or more safety factors. The method includes applying a device to a patient's epidermis prior to a treatment procedure. The device can include a transparent hydrogel layer that is pre-cooled and, in some instances, a transparent indicator layer with attached and/or embedded indicator particles covering at least a portion of a side of the transparent hydrogel layer. The method also includes delivering the treatment procedure to the patient's epidermis through the device. The indicator particles change in appearance upon application of the treatment and at least one of the transparent indicator layer and the transparent hydrogel layer act as a barrier to particles ejected from the patient's skin due to the treatment procedure. The transparent hydrogel layer achieves at least one of cooling the patient's epidermis, controlling pain in the patient's epidermis, and protecting the patient's epidermis to achieve the one or more safety factors.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a diagram illustrating a system that achieves at least one of skin cooling, pain management, laser plume control, and proper application of treatment during a treatment procedure according to an aspect of the present disclosure;

FIGS. 2-4 are side-view diagrams illustrating different configurations of the multifunctional skin patch shown in FIG. 1;

FIGS. 6 and 7 are top-view diagrams showing how the multifunctional skin patch changes in appearance upon application of the treatment procedure through the multifunctional skin patch; and

FIG. 8 is a process flow diagram illustrating a method for achieving at least one of skin cooling, pain management, laser plume control, and proper application of treatment during a treatment procedure according to another aspect of the present disclosure.

### DETAILED DESCRIPTION

### Definitions

Unless defined otherwise, all technical and scientific terms used herein have the same meaning as is commonly understood by one of skill in the art to which the present disclosure pertains.

In the context of the present disclosure, the singular forms “a,” “an” and “the” can also include the plural forms, unless the context clearly indicates otherwise.

The terms “comprises” and/or “comprising,” as used herein, can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure.

As used herein, the term “skin patch” can refer to a device that can be applied to a patient's skin before, during, and/or after a dermatology application and used before, during, and/or after the dermatology application. In some instances, the skin patch may be disposable.

As used herein, the term “multifunctional” can refer to being able to perform one or more functions. As an example, a multifunctional skin patch can be able to achieve one or more safety factors before, during, and/or after a dermatology application.

As used herein, the term “dermatology application” can refer to a treatment procedure undertaken related to or undertaken through a patient's skin.

As used herein, the term “treatment procedure” can refer to a course of action intended to achieve a result (e.g., related to a patient's skin). In terms of dermatology, treatment procedures can include, but are not limited to, treatment of blood vessel pathologies (as in congenital vascular lesions and telangiectasias), elimination of brown spots (as in nevi and lentigines), hair removal, tattoo removal, skin resurfacing, treatment of scars, etc.

As used herein, the term “treatment device” can refer to an article, instrument, apparatus, or machine that is used to facilitate a treatment procedure. The treatment device can be used before, during, and/or after the treatment procedure. For example, in dermatology, the treatment device can be a microneedle device, an injection device, an energy delivery device, or the like.

As used herein, the term “energy delivery device” can refer to something adapted to deliver energy (e.g., electromagnetic energy, sound energy, or the like) during a treatment procedure. In some instances, the energy delivery device can also generate the electromagnetic energy. Examples of energy delivery devices include a light delivery device, a laser device, an ultrasound delivery device, an x-ray delivery device, etc.

As used herein, the term “safety factor” can refer to a circumstance, fact, and/or influence that contributes (positively and/or negatively) to the condition of being protected from or unlikely to cause danger, risk, or injury. Examples of safety factors include skin cooling, pain management, laser plume control, proper application of the treatment, and the like.

As used herein, the term “plume” can refer to one or more of vapors, smoke, and/or particulate debris produced during a treatment procedure. As an example, the plume created during a laser procedure can be hazardous to a person's health (e.g., a patient, a medical professional, anyone involved in the treatment procedure, or the like). The terms “plume” and “laser plume” can be used interchangeably herein.

As used herein, the term “substantially” can refer to a majority of something being in a condition. In some instances, the majority can be 50% or more. In other instances, the majority can be 55, 60, 65, 70, 75, 89, 85, 90, 95, 97, or 99% or more.

As used herein, the terms “subject” and “patient” can be used interchangeably and refer to any warm-blooded organism including, but not limited to, a human being, a pig, a rat, a mouse, a dog, a cat, a goat, a sheep, a horse, a monkey, an ape, a rabbit, a cow, etc.

### Overview

Dermatology applications include treatment procedures undertaken related to a patient's skin. Often, such treatment procedures include the use of lasers. When using lasers, one or more safety factors must be considered, including skin cooling, pain management, plume control, and proper application of the treatment. The present disclosure describes a disposable multifunctional skin patch that can be applied to the skin undergoing a treatment procedure (before, during, and/or after the treatment procedure). In some instances, the treatment procedure can be conducted through the multifunctional skin patch. The multifunctional skin patch can achieve at least one of skin cooling (e.g., through contact cooling), pain management, plume control, and proper application of the treatment (e.g., through use of an indicator).

The multifunctional skin patch includes a transparent hydrogel layer (which can be pre-cooled) that can be applied to a patient's skin (before, during, and/or after the treatment procedure). The transparent hydrogel layer can cool and/or control pain within the patient's skin before, during and/or after the treatment procedure. In some instances, the multifunctional skin patch can also include a transparent indicator layer (covering at least a portion of the transparent hydrogel layer) with dispersed indicator particles attached thereto and/or embedded within. The transparent hydrogel layer can also act as a shield to facilitate laser plume control. The indicator particles are configured to change in appearance upon application of treatment procedure through the transparent indicator layer to ensure proper application of the treatment.

### Systems

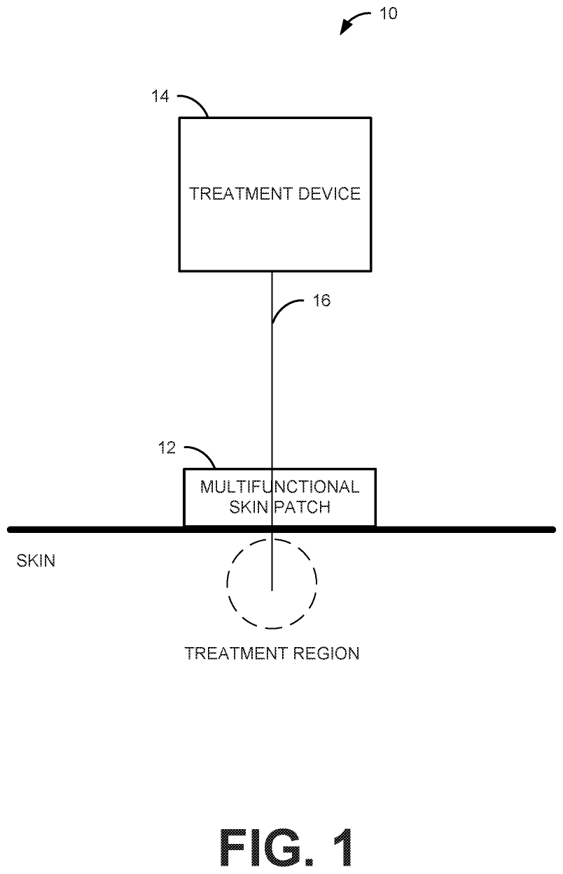

FIG. 1 illustrates a system 10 that facilitates a dermatology procedure, including a multifunctional skin patch 12 and a treatment device 14. The multifunctional skin patch 12 can be applied to a patient's skin to perform one or more functions (e.g., related to one or more safety factors). The treatment device 14 can perform a treatment procedure on the patient's skin. The multifunctional skin patch 12 can be applied to the patient's skin before, during, and/or after the treatment procedure. In some instances, the treatment procedure can be conducted by the treatment device 14 through the multifunctional skin patch 12.

The treatment device 14 can include one or more of a microneedle device, an injection device, and/or an energy delivery device (delivering light energy, x-ray energy, ultrasound energy, other electromagnetic energy, or the like). The multifunctional skin patch 12 can achieve at least one safety factor before, during, and/or after the treatment is conducted. The treatment can be provided according to a treatment procedure as part of a dermatology application. The treatment procedure can include, for example, treatment of blood vessel pathologies (as in congenital vascular lesions and telangiectasias), elimination of brown spots (as in nevi and lentigines), hair removal, tattoo removal, skin resurfacing, treatment of scars, etc. The treatment procedure, in some instances, can also include drawing blood, receiving a vaccination, conducting a test (e.g., a tuberculosis skin test), or the like.

As noted, the multifunctional skin patch 12 can achieve at least one safety factor before, during, and/or after the treatment procedure is conducted. The safety factor can be at least one of skin (or epidermal) cooling, pain management, plume control, and proper application of treatment during the treatment procedure. In some instances, the multifunctional skin patch 12 can achieve at least two such safety factors. In further instances, the multifunctional skin patch 12 can achieve at least three such safety factors. In still further instances, the multifunctional skin patch 12 can achieve at least four (or more) such safety factors.

In some instances, the multifunctional skin patch 12 can achieve skin (or epidermal) cooling. The skin can be cooled before, during, and/or after the treatment procedure. This cooling can be via contact cooling. By cooling the skin, the multifunctional skin patch 12 can prevent skin damage in the instant that the therapy is delivered through the skin. This can be especially important during a treatment procedure that is photothermal in nature, such as a laser procedure, which involves the interaction between light and a target chromophore. Although thermal confinement is achieved at the target chromophore when the parameters of the treatment device 14 are chosen correctly, there is still heating of the surrounding tissue, which can be due to conduction (where some heat flows from the target chromophore to the surrounding tissue), undesired targeting of a chromophore that absorbs the same wavelength as the desired chromophore, or the like, which can damage the skin (or epidermis) and/or cause dyspigmentation. Previous solutions involve using ice packs, cold air, or spray cooling. However, the multifunctional skin patch 12 is superior to these previous solutions at least because the multifunctional skin patch 12 can be used to provide the cooling for several minutes (or longer) during the course of the treatment procedure.

In other instances, the multifunctional skin patch 12 can achieve pain management (or mitigation). The pain management can be achieved by cooling the skin before, during, and/or after the treatment procedure. It is well known that treatment procedures may cause some amount of pain. Previous solutions include local anesthesia and ice packs. However, the multifunctional skin patch 12 is superior to these previous solutions at least because the multifunctional skin patch 12 can provide cooling that can be used to manage the pain during the course of the treatment procedure and/or after the treatment procedure.

In still other instances, the multifunctional skin patch 12 can manage the plume associated with the delivery of the therapy. Many laser treatments can create a hazardous plume. When the treatment procedure uses a laser, for example, a plume of particles can fly off the skin, some which may be hazardous. For example, the plume can include water, intact fragments of skin, vaporized skin, intact hair, vaporized hair, other biological particles, other nanoparticles, or the like. The multifunctional skin patch 12 can catch the plume and embed the plume therein. Previous solutions include the use of suction or barriers to manage the plume. However, the multifunctional skin patch 12 is superior to the previous solutions because the multifunctional skin patch 12 can embed the plume therein without letting the plume escape into the atmosphere.

In further instances, the multifunctional skin patch 12 can ensure proper application of treatment. The multifunctional skin patch 12 can include an indicator material that can cause the multifunctional skin patch 12 to change in appearance where the treatment has occurred. This helps the person conducting the treatment procedure both avoid skipping areas and avoid irradiating the same area multiple times. Although an indicator dye has been used previously to ensure proper application of skin resurfacing treatment, the multifunctional skin patch 12 can expand the previous use of indicator dyes to encompass more treatment procedures beyond skin resurfacing.

The treatment procedure can be conducted by the treatment device 14 through the multifunctional skin patch 12. In other words, the treatment device 14 (e.g., one or more of a microneedle device, an injection device, and/or an energy delivery device) can pass the treatment 16 through the multifunctional skin patch 12 and into the patient's skin (or epidermis) to a treatment region. The treatment device 14 can, in some instances, generate the treatment 16. In some instances, at least a portion of the multifunctional skin patch 12 can be optically transparent or substantially optically transparent (before the treatment procedure is conducted). The multifunctional skin patch 12 can be a single use, disposable device.

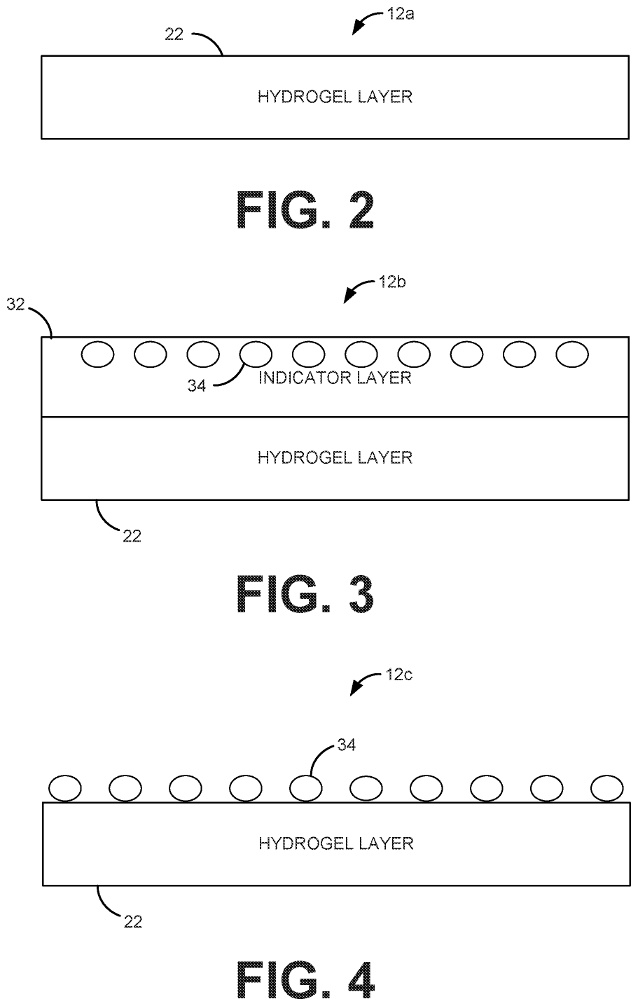

As shown in FIG. 2-4, variations of the multifunctional skin patch 12a (FIG. 2), 12b (FIG. 3), 12c (FIG. 4) can include a hydrogel layer 22, which can be transparent or substantially transparent, semisolid, and can be applied to a patient's skin before, during, or after the treatment procedure is conducted. In some instances, the hydrogel layer 22 can include one or more clarifying agents. The hydrogel layer 22 can be made of any of a number of different hydrogel materials, like gelatin, agarose, etc. Flexibility and/or consistency of the hydrogel layer 22 can be adjusted by varying the concentration of the gel and/or thickness of the gel. The hydrogel layer 22 can be precooled (e.g., by placing in a refrigerator or freezer for a period before use) to regulate temperature and/or control pain in the patient's skin (or epidermis) during and/or after the treatment procedure. For example, the hydrogel layer 22 can cool the patient's skin and/or control the pain by contact cooling. In addition, the hydrogel layer 22 can be configured to create a shield against any plume(s) generated by the treatment procedure.

As shown in FIGS. 3 and 4, the multifunctional skin patch 12b (FIG. 3), 12c (FIG. 4) can include an indicator layer 32 with indicator particles 34 (invisible or barely visible) distributed therein. The indicator layer 32 can be a transparent or substantially transparent sheet, for example, of polyester, acetate, or the like. The indicator particles 34 may be dispersed in a variety of ways, including on an upper surface of the indicator layer 32 (FIG. 3), attached to an outer surface of the indicator layer 32 (not shown), and/or attached to an outer surface surface of the hydrogel layer 22 (FIG. 4, for example, the indicator particles 34 can be within another material, such as release particles, and spray coated on the hydrogel layer 22). The indicator particles 34 also can be embedded within the hydrogel layer 22 (not shown, these particles, in some instances, can be enclosed within other particles, such as release particles, which can be triggered to release the indicator particles 34 into the hydrogel). The indicator layer 32 and/or the indicator particles 34 can be attached to and/or embedded within the hydrogel layer 22 before and/or after the hydrogel layer 22 is pre-cooled. In some instances, the indicator particles 34 can be contained within a release mechanism that can be triggered (e.g., by the treatment procedure) to release the indicator particles 34.

In FIG. 3, the indicator layer 32 covers the entirety of the hydrogel layer 22, but need only cover at least a portion of the hydrogel layer 22. No air bubbles should exist between the indicator layer 32 and the hydrogel layer 22, at least because any air bubbles can result in undesired reflections and a corresponding decrease in treatment efficacy. The multifunctional skin patch 12b can be constructed to avoid developing air bubbles between the indicator layer 32 and the hydrogel layer 22. As one example, a thin layer of a somewhat volatile solution (that is easily evaporable, such as isopropyl alcohol) can be placed on top of an already-formed semisolid hydrogel and the indicator layer 32 can be placed atop the somewhat volatile substance (which can evaporate or be actively removed); the liquid nature of the somewhat volatile solution makes air bubbles less likely to form. As another example, the indicator layer 32 can be attached to the hydrogel layer 22 while the hydrogel is still in liquid form, thereby minimizing the formation of air bubbles. As a further example, the indicator layer 32 can be attached to the hydrogel layer 22 using an adhesive (e.g., superglue) and the layers can be pressed together to remove air bubbles. In some instances, the indicator layer 32 can be hydrophobic.

The indicator layer 32 can create a shield or barrier against the plume (e.g., where particles ejected from the patient's skin can become embedded in the indicator layer 32) and/or change in appearance upon application of the treatment procedure therethrough. The indicator layer 32 can be constructed of materials that do not have high thermal conductivity. In some instances, water must be excluded from the indictor layer (or, in other words, the indictor layer at must be non-aqueous) because water has a high thermal conductivity. In this example, if water were contained in the indicator layer 32, the indicator particles 34 would be cooled too quickly and would not change the appearance of the indicator layer 32. In fact, the indicator particles 34 in the presence of water would effectively remain invisible. In FIG. 4, the indicator particles 34 (or ink particles contained within a hydrophobic layer) are attached to the surface of the hydrogel layer 22. Alternatively, the indicator particles 34 can be attached to the outer surface of the indicator layer 32.

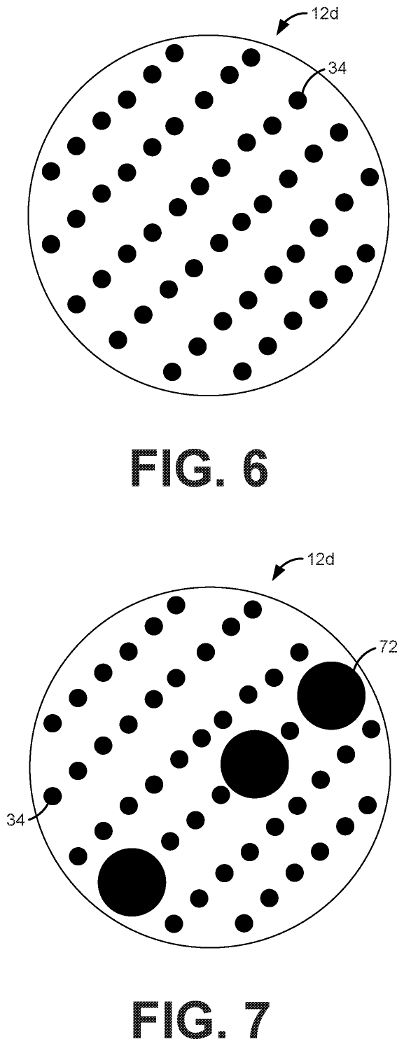

In any form, the indicator particles 34 (or the indicator particles 34 within release mechanisms) can be configured to change a perceivable feature of the indicator layer 32 and/or the hydrogel layer 22 upon application of the treatment procedure therethrough. For example, the perceivable feature can be a refractive index, a color, an opacity, or the like. Operation of the indicators is shown schematically in the top view of a multifunctional skin patch 12d, shown in FIGS. 6 and 7. It should be noted that FIGS. 6 and 7 are not drawn to scale and the color does not represent reality (e.g., the indicator particles are represented as black dots, when the indicator particles are actually invisible or substantially invisible).

FIG. 6 represents the multifunctional skin patch 12d before the therapy procedure is conducted therethrough. FIG. 7 represents the multifunctional skin patch 12d after the treatment procedure has been conducted through three portions, resulting in visible changes 72. In this example, the indicator particles 34 are located in predefined positions on the multifunctional skin patch 12d. When the treatment procedure is conducted through a portion of the multifunctional skin patch 12d (e.g., a laser is shined through), one or more indicator particles 34 in the area undergoing treatment experiences a change in a perceivable feature 72 (e.g., a color change, an opacity change, a refractive change, or the like). As the treatment procedure is conducted through more portions of the multifunctional skin patch 12d, a corresponding number of portions of the multifunctional skin patch 12d will experience the change in the perceivable feature 72 (e.g., the treatment procedure has been conducted through three portions of the multifunctional skin patch 12 in FIG. 7). Areas that have not yet experienced the treatment procedure can be readily identified.

### Methods

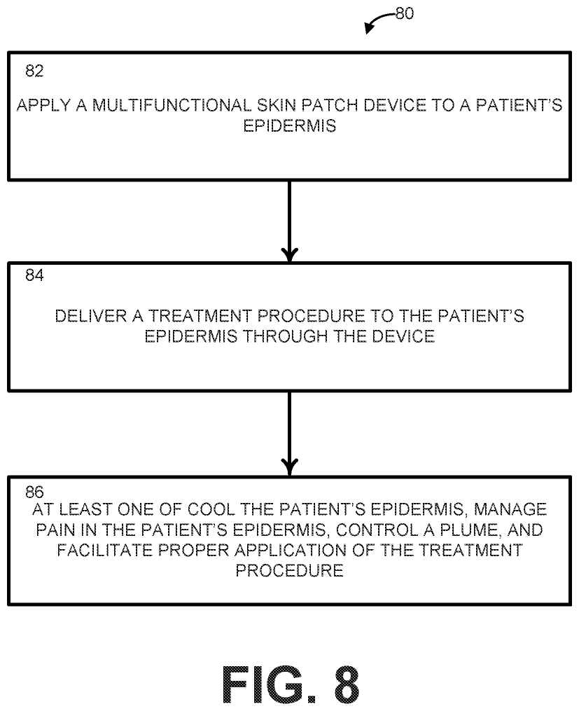

Another aspect of the present disclosure can include a method 80 (FIG. 8) for achieving at least one of skin cooling, pain management, laser plume control, and proper application of treatment during a treatment procedure. The method 80 can be performed, for example, by the system of FIG. 1 using a multifunctional skin patch, for example, as shown in FIGS. 2-7.

The method 80 is illustrated as a process flow diagram with flowchart illustrations. For purposes of simplicity, the method 80 shown is and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the method 80.

At 82, a multifunctional skin patch device (e.g., shown in shown in FIGS. 2-9) can be applied to a patient's skin (or epidermis). For example, the device can include a transparent hydrogel layer that is pre-cooled and a transparent indicator layer with dispersed indicator particles covering at least a portion of a side of the transparent hydrogel layer. The device can be applied to the patient's skin (or epidermis) prior to conducting a treatment procedure and/or while the treatment procedure is being conducted.

At 84, the treatment procedure can be delivered to the patient's skin (or epidermis) through the device. The treatment procedure can be a dermatology procedure, such as treatment a blood vessel pathology, elimination of brown spots, permanent hair removal, tattoo removal, skin resurfacing, treatment of a scar, or the like. In some instances, the treatment procedure can employ one or more energy delivery device (delivering light energy, x-ray energy, other electromagnetic energy, or the like), one or more injection mechanisms, one or more microneedles, or the like to deliver the treatment procedure through the device. As an example, the one or more energy delivery device(s) can be one or more lasers configured to generate a plurality of laser pulses for the treatment procedure.

At 86, the device achieves at least one of cooling the patient's skin (or epidermis), managing pain perceived by the patient in the patient's skin (or epidermis), controlling a laser plume from the treatment procedure, and facilitating proper application of the treatment procedure. The indicator particles change in appearance upon application of the treatment and at least one of the transparent indicator layer and the transparent hydrogel layer act as a barrier to particles ejected from the patient's skin (or epidermis) due to the treatment procedure. The transparent hydrogel layer at least one of cools the patient's skin (or epidermis), controls pain in the patient's skin (or epidermis), and protects the patient's skin (or epidermis). The positive consequences caused by the device can occur before, during, and/or after the treatment is delivered (e.g., for at least three minutes after the treatment is delivered).

From the above description, those skilled in the art will perceive improvements, changes and modifications. Such improvements, changes, and modifications are within the skill of one in the art and are intended to be covered by the appended claims.

What is claimed is:

1. A system comprising:
a transparent hydrogel configured to be pre-cooled and applied to a portion of a patient's skin prior to a treatment procedure to cool and/or control pain within the portion of the patient's skin during and/or after the treatment procedure; and
a treatment device configured to apply the treatment procedure through the transparent hydrogel to the patient's skin, wherein the treatment device comprises a microneedle device, an injection device, and/or an energy delivery device.

2. The system of claim 1, wherein an indicator and/or a release mechanism comprising the indicator is embedded within and/or attached to the transparent hydrogel before and/or after the transparent hydrogel is pre-cooled, wherein the indicator is configured to change a perceivable feature upon application of the treatment procedure through the transparent hydrogel.

3. The system of claim 1, wherein the treatment device comprises a laser energy device, and
wherein the transparent hydrogel is configured to create a shield for a laser plume generated by the treatment procedure.

4. The system of claim 1, further comprising a transparent indicator layer, atop the transparent hydrogel, configured to at least one of to create a shield for a laser plume generated by the treatment procedure and change in appearance upon application of the treatment procedure.

5. A device comprising:
a transparent indicator layer with dispersed indicator particles attached thereto and/or embedded within, wherein the indicator particles are configured to change in appearance upon application of treatment procedure through the transparent indicator layer; and
a transparent hydrogel layer to be applied to a patient's skin prior to application of the treatment procedure through the device cool and/or control pain within the portion of the patient's skin during and/or after the treatment procedure, wherein the transparent hydrogel layer is pre-cooled,
wherein the transparent indicator layer covers at least a portion of the transparent hydrogel layer.

6. The device of claim 5, wherein the change in appearance assists an operator in appropriate spacing of application of the treatment procedure, wherein the treatment procedure comprises application of at least two laser pulses.

7. The device of claim 5, wherein each of the indicator particles comprises a particle size less than or equal to a maximum particle size chosen based on a length of a laser pulse used during the treatment procedure.

8. The device of claim 5, wherein the transparent hydrogel layer is pre-cooled to protect the patient's epidermis from thermal injury by contact cooling.

9. The device of claim 8, wherein the contact cooling controls pain felt by the patient due to the application of a laser pulse used during the treatment procedure.

10. The device of claim 9, wherein the contact cooling lasts for at least three minutes after the application of the laser pulse.

11. The device of claim 5, wherein the transparent indicator layer and/or the transparent hydrogel layer is configured to mitigate a lase plume generated due to the application of a laser pulse used during the treatment procedure.

12. The device of claim 11, wherein the transparent indicator layer and/or the transparent hydrogel layer acts as a barrier to particles ejected from the patient's skin due to the application of the laser pulse.

13. The device of claim 12, wherein the particles ejected from the patient's skin due to the application of the laser pulse become embedded within the transparent indicator layer and/or the transparent hydrogel layer.

14. A method comprising:
applying a device, comprising a transparent hydrogel layer that is pre-cooled and a transparent indicator layer with attached and/or embedded indicator particles covering at least a portion of a side of the transparent hydrogel layer, to a patient's epidermis prior to a treatment procedure;
delivering the treatment procedure to the patient's epidermis through the device, wherein the indicator particles change in appearance upon application of the treatment and at least one of the transparent indicator layer and the transparent hydrogel layer act as a barrier to particles ejected from the patient's skin due to the treatment procedure, and wherein the transparent hydrogel layer at least one of cools the patient's epidermis, controls pain in the patient's epidermis, and protects the patient's epidermis.

15. The method of claim 14, wherein the treatment procedure is a dermatology procedure,
wherein the dermatology procedure comprises treatment of at least one blood vessel, elimination of brown spots, permanent hair removal, tattoo removal, skin resurfacing, or treatment of a scar.

16. The method of claim 14, wherein the treatment procedure comprises a laser treatment, and
wherein the transparent hydrogel layer is used to cool at least a portion of the patient's epidermis as the laser treatment is delivered.

17. The method of claim 16, wherein the transparent hydrogel layer is used to cool the at least the portion of the patient's epidermis for a time after the laser treatment is delivered.

18. The method of claim 17, wherein the time is at least three minutes after the laser treatment is delivered.

19. The method of claim 16, wherein the laser treatment comprises a plurality of laser pulses.

20. The method of claim 19, wherein the indicator particles are configured to increase an opacity of the transparent indicator layer by changing a perceivable feature with application of at least one of the plurality of laser pulses to an area of the transparent indicator layer.
